(19) United States
(12) Patent Application Publication
(10) Pub. No.: US 2010/0063842 A1
(43) Pub. Date: Mar. 11, 2010

(54) SYSTEM AND METHODS FOR INDICATING AN IMAGE LOCATION IN AN IMAGE STACK

(75) Inventors: Joseph Carroll, Arlington Heights, IL (US); Benjamin D. Novatzky, Oak Park, IL (US); Kengo Baba, Evanston, IL (US); John Yan, Libertyville, IL (US)

Correspondence Address: HANLEY, FLIGHT & ZIMMERMAN, LLC, 150 S. WACKER DRIVE, SUITE 2100, CHICAGO, IL, 60606, US

(73) Assignee: GENERAL ELECTRIC COMPANY, Schenectady, NY (US)

(21) Appl. No.: 12/206,232

(22) Filed: Sep. 8, 2008

(51) Int. Cl. A61B 5/00

(52) U.S. Cl. 705/3

(57) ABSTRACT

System and methods for indicating an image location in an image stack are disclosed. An example method includes generating an index of indications of medical image locations in a medical image stack including a first indication of a first medical image location in a medical image stack and a second indication of a second medical image location in the medical image stack, displaying in a user interface the first medical image and the first indication of the first medial image location within the medical image stack, receiving a scroll input and scroll through the index displaying indications of medical image locations in the image stack until the second indication of the second medical image is displayed, receiving a selection of the second indication of the second medical image, and displaying in the user interface the second medical image and the second indication of the second medical image location within the medical image stack.

### FIELD OF THE DISCLOSURE

This disclosure relates generally to dynamic display information and more particularly, to system and methods for indicating an image location in an image stack in a picture archiving and communication system.

### BACKGROUND OF THE INVENTION

A clinical or healthcare environment is a crowded, demanding environment that would benefit from organization and improved ease of use of imaging systems, data storage systems, and other equipment used in the healthcare environment. A healthcare environment, such as a hospital or clinic, encompasses a large array of professionals, patients, and equipment. Personnel in a healthcare facility must manage a plurality of patients, systems, and tasks to provide quality service to patients. Healthcare personnel may encounter many difficulties or obstacles in their workflow.

Healthcare environments, such as hospitals or clinics, include clinical information systems, such as hospital information systems (HIS) and radiology information systems (RIS), and storage systems, such as picture archiving and communication systems (PACS). Information stored may include patient medical histories, imaging data, test results, diagnosis information, management information, and/or scheduling information, for example. The information may be centrally stored or divided at a plurality of locations. Healthcare practitioners may desire to access patient information or other information at various points in a healthcare workflow. For example, during surgery, medical personnel may access patient information, such as images of a patient's anatomy, which is stored in a medical information system. Alternatively, medical personnel may enter new information, such as history, diagnostic, or treatment information, into a medical information system during an ongoing medical procedure.

A reading, such as a radiology or a cardiology procedure reading is a process of a healthcare practitioner, such as a radiologist or a cardiologist, viewing digital images of a patient. The practitioner performs a diagnosis based on a content of the diagnostic images and reports on results electronically (e.g., using dictation or otherwise) or on paper. The practitioner, such as a radiologist or cardiologist, typically uses other tools to perform diagnosis. Some examples of other tools are prior and related prior (historical) exams and their results, laboratory exams (such as blood work), allergies, pathology results, medication, alerts, document images, and other tools.

Picture archiving and communication systems (PACS) connect to medical diagnostic imaging devices and employ an acquisition gateway (between the acquisition device and the PACS), storage and archiving units, display workstations, databases, and sophisticated data processors. These components are integrated together by a communication network and data management system. A PACS has, in general, the overall goals of streamlining health-care operations, facilitating distributed remote examination and diagnosis, and improving patient care.

A typical application of a PACS system is to provide one or more medical images for examination by a medical professional. For example, a PACS system can provide a series of x-ray images to a display workstation where the images are displayed for a radiologist to perform a diagnostic examination. Based on the presentation of these images, the radiologist can provide a diagnosis. For example, the radiologist can diagnose a tumor or lesion in x-ray images of a patient's lungs.

Typically, data stored in a PACS is stored as Digital Imaging and Communications in Medicine (DICOM) data. DICOM is a standard for image and information transmission. DICOM relates to the transfer of electronic data between medical diagnostic and imaging systems. The DICOM protocol may be employed in communication between medical devices and PACS. The DICOM standard enumerates a command set, data formats, interface specifications, communication protocols, and command syntax. However, the DICOM standard does not specify details of implementation. DICOM sets forth Information Objects (types of data, such as computerized tomography, magnetic resonance, x-ray, ultrasound, etc.), Service Classes (actions with data, such as send, receive, print, etc.), and data transmission protocols. The Service Class User (SCU) protocol governs use of the DICOM service. The Service Class Provider (SCP) protocol governs the provider of the DICOM service.

Current PACS systems use general techniques known as hanging protocols to format display or layout of images. Hanging protocols allow a user to display images based on modality, anatomy, and procedure. Hanging protocols present a perspective or view to a user, such as a radiologist. Images may be grouped according to characteristics such as DICOM series or series number.

Additionally, PACS systems attempt to prepare images for viewing by users by applying a series of processing steps or functions included in a Default Display Protocol (DDP). A DDP is a default workflow that applies a series of image processing functions to image data to prepare the image data for presentation to a user on a particular monitor configuration. DDPs typically include processing steps or functions that are applied before any diagnostic examination of the images. A DDP may be based on a type of imaging modality used to obtain the image data, for example. In general, a DDP attempts to present image data in a manner most useful to many users.

With increasing volumes of examinations and images, a reduction of radiologists and mounting pressures on improved productivity, radiologists and other healthcare personnel are in need of image processing or display workflow enhancements that alleviate rote, repetitive tasks. Currently, radiologists or healthcare personnel need to scroll through multiple images on workstations. The images may be part of hundreds of images in a stack generated during a cardiology imaging procedure. The display protocols and related functionalities of the workstations require healthcare personnel to scroll through the images one at a time or require the healthcare personnel to know which images are in which locations of the stack. This manual scrolling is inefficient if healthcare personnel are searching for a specific image or specific images in different parts of the stack. The inefficient manner of scrolling and searching causes healthcare personnel to spend extra time searching for images and comparing information between two or more images. This extra time reduces their ability to perform other tasks such as treating patients or examining medical information for other patients. Additionally, the manual searching, scrolling, and remembering image locations creates a possibility for healthcare personnel to form an inaccurate diagnosis from the images.

### BRIEF SUMMARY OF THE INVENTION

Example system and methods for indicating an image location in an image stack are described. In one example, a method involves indicating a medial image location in an image stack. In particular, generating an index of indications of medical image locations in a medical image stack including a first indication of a first medical image location in a medical image stack and a second indication of a second medical image location in the medical image stack. Next, displaying in a user interface the first medical image and the first indication of the first medial image location within the medical image stack, receiving a scroll input and scrolling through the index displaying indications of medical image locations in the image stack until the second indication of the second medical image is displayed, and receiving a selection of the second indication of the second medical image. Then, displaying in the user interface the second medical image and the second indication of the second medical image location within the medical image stack.

Another example method includes generating an index of indications of medical image locations in a medical image stack of a primary medical image including a first indication of a first medical image location in a medical image stack and a second indication of a second medical image location in the medical image stack. Then, receiving a selection of the location on the primary medical image of the first medical image and displaying in a user interface the first medical image and the first indication of the first medial image location within the primary image. Next, receiving a scroll input and scrolling through the index displaying indications of medical image locations in the primary image until the second indication of the second medical image is displayed, receiving a selection of the second indication of the second medical image, and displaying in the user interface the second medical image and the second indication of the second medical image location within the primary image.

In another example, a system involves an index generator to generate an index of indications of medical image locations in a medical image stack including a first indication of a first medical image location in a medical image stack and a second indication of a second medical image location in the medical image stack, an input receiver to receive an amount of scrolling and to receive a selected indication, and a processor to scroll through the index by the received amount of scrolling, to send to a user interface indications of medical image locations in the image stack as the indications are scrolled, and to send to the user interface a medical image corresponding to the received selected indication.

In some example implementations, the example system further includes a display system including the user interface to display indications of medical image locations in the stack including the first indication and the second indication and displays medical images including the first medical image and the second medical image. Additionally, in certain implementations, the example system further includes a temporary memory to store at least one of the medical image stack, the index, or the indications of medical image locations in the medical image stack and a thumbnail generator to generate thumbnail images corresponding to medical images in the medical image stack.

Certain implementations provide a machine accessible medium having instructions stored thereon for execution on a processor. The instructions that, when executed, cause a machine to generate an index of indications of medical image locations in a medical image stack including a first indication of a first medical image location in a medical image stack and a second indication of a second medical image location in the medical image stack, display in a user interface the first medical image and the first indication of the first medial image location within the medical image stack, scroll through the index displaying indications of medical image locations in the image stack until the second indication of the second medical image is displayed, select the second indication of the second medical image, and display in the user interface the second medical image and the second indication of the second medical image location within the medical image stack.

### BRIEF DESCRIPTION OF SEVERAL VIEWS OF THE DRAWINGS

FIG. 1 shows a picture archiving and communication system including an example scroll cine manager.

FIG. 2 shows a functional diagram of the example scroll cine manager of FIG. 1.

FIG. 3 shows an example user interface displaying indications of image location in a stack by representative thumbnail images adjacent to a scale managed by the example scroll cine manager of FIG. 2.

FIG. 4 shows the example user interface of FIG. 3 displaying an indication in a preview box of an image location in a stack managed by the scroll cine manager of FIG. 2.

FIG. 5 shows the example user interface of FIG. 3 displaying an indication of an image location in a stack managed by the scroll cine manager of FIG. 2.

FIG. 6 shows an example configuration interface for the scroll cine manager 150 of FIG. 2.

FIGS. 7 and 8 are flowcharts representative of example machine accessible instructions that may be executed by, for example, a processor to implement any portion or all of the example scroll cine manager of FIG. 2.

FIG. 9 is a schematic illustration of an example processor platform that may be used and/or programmed to implement the example scroll cine manager of FIG. 2 and/or to carry out indicating an image location in an image stack and/or the example machine accessible instructions of FIGS. 7 and/or 8 to implement any or all of the example system and methods described herein.

The foregoing summary, as well as the following detailed description of certain implementations of the example scroll cine manager, will be better understood when read in conjunction with the appended drawings. For the purpose of illustrating the scroll cine manager, certain implementations are shown in the drawings. It should be understood, however, that the example scroll cine manager is not limited to the arrangements and instrumentality shown in the attached drawings.

### DETAILED DESCRIPTION OF THE INVENTION

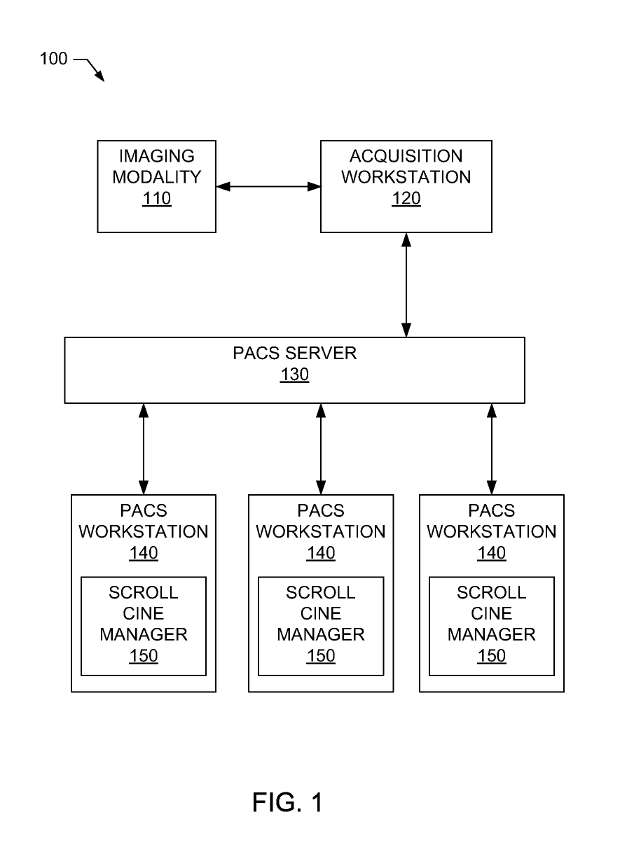

FIG. 1 illustrates a Picture Archiving and Communication System (PACS) 100 including an example scroll cine manager 150. Additionally, the PACS system 100 includes an imaging modality 110, an acquisition workstation 120, a PACS server 130, and one or more PACS workstations 140. The system 100 may include any number of imaging modalities 110, acquisition workstations 120, PACS server 130 and PACS workstations 140 and is not in any way limited to the embodiment of system 100 illustrated in FIG. 1. The components of the system 100 may communicate via wired and/or wireless communication, for example, and may be separate systems and/or integrated to varying degrees, for example.

In operation, the imaging modality 110 obtains one or more images of a patient anatomy. The imaging modality 110 may include any device capable of capturing an image of a patient anatomy such as a medical diagnostic imaging device. For example, the imaging modality 110 may include an X-ray imager, ultrasound scanner, magnetic resonance imager, or the like. Image data representative of the image(s) is communicated between the imaging modality 110 and the acquisition workstation 120. The image data may be communicated electronically over a wired or wireless connection, for example.

In an embodiment, the acquisition workstation 120 may apply one or more preprocessing functions, for example, to the image data in order to prepare the image for viewing on a PACS workstation 140. For example, the acquisition workstation 120 may convert raw image data into a DICOM standard format or attach a DICOM header. Preprocessing functions may be characterized as modality-specific enhancements, for example (e.g., contrast or frequency compensation functions specific to a particular X-ray imaging device), applied at the beginning of an imaging and display workflow. The preprocessing functions differ from processing functions applied to image data in that the processing functions are not modality specific and are instead applied at the end of the imaging and display workflow (for example, at a display workstation 140).

The image data may then be communicated between the acquisition workstation 120 and the PACS server 130. The image data may be communicated electronically over a wired or wireless connection, for example.

The PACS server 130 may include computer-readable storage media suitable for storing the image data for later retrieval and viewing at a PACS workstation 140. The image data may be stored as a group of images (e.g., a stack of images) from one or more instances of images generated by the imaging modality 110. The PACS server 130 may also include one or more software applications for additional processing and/or preprocessing of the image data by one or more PACS workstations 140.

One or more PACS workstations 140 are capable of or configured to communicate with the server 130. The PACS workstations 140 may include a general purpose processing circuit, a PACS server 130 interface, a software memory, and/or an image display monitor, for example. The PACS server 130 interface may be implemented as a network card connecting to a TCP/IP based network, but may also be implemented as a parallel port interface, for example.

The PACS workstations 140 may retrieve or receive image data from the server 130 for display to one or more users. For example, a PACS workstation 140 may retrieve or receive image data representative of a computed radiography (CR) image of a patient's chest. A radiologist or user may then examine the image for any objects of interest, such as tumors, lesions, etc., for example.

The PACS workstations 140 may also be capable of or configured to apply processing functions to image data. For example, a user may desire to apply processing functions to enhance features within an image representative of the image data. Processing functions may therefore adjust an image of a patient anatomy in order to ease a user's diagnosis of the image. Such processing functions may include any software-based application that may alter a visual appearance or representation of image data. For example, a processing function can include any one or more of flipping an image, zooming in an image, panning across an image, altering a window and/or level in a grayscale representation of the image data, and altering a contrast and/or brightness an image.

In an embodiment, the PACS system 100 may provide one or more perspectives for viewing images and/or accessing applications at a PACS workstation 140. Perspectives may be provided locally at the PACS workstation 140 and/or remotely from the PACS server 130. In operation, for example, a user, such as a radiologist, selects a set of images, such as screening mammogram images, chest screening images and/or other computed radiography (CR), digital radiography (DR), and/or digital x-ray (DX) screening images, to review at a PACS workstation 140. The images may be displayed in a default perspective and/or a customized perspective, for example.

The PACS workstations 140 may retrieve or receive image data from the PACS server 130 for display to one or more users. For example, a PACS workstation 140 may retrieve or receive image data representative of a computed radiography image of a patient's chest. A radiologist may then examine the image as displayed on a display device for any objects of interest such as, for example, tumors, lesions, etc.

The PACS workstations 140 are also capable of or configured to retrieve and/or receive one or more hanging protocols from server 130. For example, a default hanging protocol may be communicated to PACS workstation 140 from server 130. A hanging protocol may be communicated between server 130 and a PACS workstation 140 over a wired or wireless connection, for example.

In general, PACS workstations 140 may present images representative of image data retrieved and/or received from server 130. PACS workstations 140 may present the images according to a hanging protocol. As described above, a hanging protocol is a set of display rules for presenting, formatting and otherwise organizing images on a display device of a PACS workstation 140. A display rule is a convention for presenting one or more images in a particular temporal and/or spatial layout or sequence. For example, a hanging protocol may include a set of computer-readable instructions (or display rules, for example) that direct a computer to display a plurality of images in certain locations on a display device and/or display the plurality of images in a certain sequence or order.

A hanging protocol may direct, for example, a PACS workstation 140 to display an anterior-posterior (AP) image adjacent to a lateral image of the same anatomy. In another example, a hanging protocol may direct PACS workstation 140 to display the AP image before displaying the lateral image. In general, a hanging protocol dictates the spatial and/or temporal presentation of a plurality of images at PACS workstation 140.

A hanging protocol may differ from a default display protocol (DDP). In general, a DDP is a default workflow that applies a series of image processing functions to image data. The image processing functions are applied to the image data in order to present an image (based on the image data) to a user. The image processing functions alter the appearance of image data. For example, an image processing function may alter the contrast level of an image.

DDPs typically include processing steps or functions that are applied before any diagnostic examination of the images. For example, processing functions may be applied to image data in order to enhance features within an image (based on the image data). Such processing functions can include any software-based application that may alter a visual appearance or representation of image data. For example, a processing function can include any one or more of flipping an image, zooming in an image, panning across an image, altering a window and/or level setting in a representation of the image data, and altering a contrast and/or brightness setting in a representation of the image data.

DDPs are usually based on a type of imaging modality used to obtain the image data. For example, image data obtained with a C-arm imaging device in general or a particular C-arm imaging device may have a same or similar DDP applied to the image data. In general, a DDP attempts to present image data in a manner most useful to many users. Conversely, applying a hanging protocol to image data does not alter the appearance of an image (based on the image data), but instead dictates how the image(s) is (are) presented, as described above.

The PACS Server 130 may store a plurality of hanging protocols and/or DDPs. The hanging protocols and/or DDPs that are stored at server 130 and have not yet been modified or customized are default hanging protocols/DDPs. A default hanging protocol and/or DDP may be selected from a plurality of default hanging protocols and/or DDPs based on any number of relevant factors such as, for example, a manual selection, a user identity, and/or pre-processing of the image data.

Specifically, a default hanging protocol and/or DDP may be selected based on a manual selection simply by communicating the default protocol once a user has selected that particular protocol. The user may make the selection, for example, at a PACS workstation 140. Selection of a hanging protocol on a PACS workstation 140 may be based on a plurality of criteria, such as a number of connected displays, a modality, an anatomy, and a procedure, for example. The hanging protocols with perspectives/views may use one or more criteria to select a protocol for display. For example, a modality, an anatomy or body part, a procedure, and/or a default view for a display configuration, may be used to select an appropriate display protocol. For example, a display protocol includes a perspective/view with multiple options depending upon monitor configuration.

Perspectives are views or layouts indicating visual component positioning and interactions between images and/or applications based on workflow, for example. Medical perspectives may be used to create a plurality of benefits for uses. Furthermore, for example, perspectives provide an ability to store or “remember” specific workflow steps. Perspectives provide a mechanism to save and display information relevant to a particular user, group, and/or function, for example.

The PACS server 130 of FIG. 1 is communicatively coupled to the example scroll cine managers 150 located within PACS workstations 140 to manage the display of medical images. While FIG. 1 shows a single scroll cine manager 150 within each respective PACS workstation 140, some PACS workstations 140 may not include a scroll cine manager 150. Alternatively, the scroll cine manager 150 may be include within the PACS 130 server such that one or more scroll cine managers 150 on the PACS server 130 may process different groups of stacks for one or more PACS workstations 140.

The scroll cine manager 150 may implement any one of the hanging protocols, perspectives and/or DDPs to display one or more medical images in a user interface displayed on the PACS workstations 140. The scroll cine manager 150 enables a user to scroll through multiple medical images (e.g., a stack of images) generated during one or more imaging procedures in the imaging modality 110. The images are generally displayed one at a time in the user interface on the PACS workstations 140. A user accesses the images in the stack by scrolling through the stack to the desired image.

Currently, scrolling through a stack of medical images can be slow because a user can only scroll through one image at a time without skipping images or has to scroll through using reference numbers of the images in the stack. These scrolling techniques provide little or no feedback before a user clicks on an image and/or a reference to the image as to what will happen when the user clicks on that image. For example, a physician viewing medical images may have hundreds to thousands of images to scroll through. The current processes require the physician to scroll through the images one at a time, which can be very slow, or know the reference number for the desired image in order to find the image in the stack.

The example scroll cine manager 150 of FIG. 1 may be enabled when a user opens a user interface to display medical images on a PACS workstation 140. Alternatively, the scroll cine manager 150 may be activated in an already open user interface by a user inputting a hotkey from an input device and/or by a user selecting an icon in the user interface corresponding to the scroll cine manager 150. Furthermore, a user defined and/or default hanging protocol (and/or perspective) may activate the scroll cine manager 150 upon a user viewing certain types of images in the user interface. Additionally, the hanging protocol and/or perspective may organize the stack of images by user defined criteria and/or by default criteria. For example, an image stack containing images generated during a computer tomography (CT) scan of a set of lungs of a person may be organized by a hanging protocol by the order in which each image was generated and/or by the depth into the set of lungs depicted in the images.

Upon activation, the example scroll cine manager 150 generates an index of indications of image locations within a stack of images. The stack of images is stored in the PACS server 130 and copied into a temporary memory for each PACS workstation 140 accessing the stack. The generation of the index of indications includes generating a small image (e.g., thumbnail image) representative of each of the corresponding larger full resolution images in the stack, generating text information describing the location of each image in the stack, and generating a scale representing the location of each image within the stack. The index provides a means for the scroll cine manager 150 to quickly display indications of image locations within the stack as a user scrolls without having to process the information associated with each image. For example, if the scroll cine manager 150 detects a user scrolls quickly corresponding to 25 images, the scroll cine manager 150 moves 25 lines in the index to the indication scrolled to be the user.

In an example implementation of the scroll cine manager 150, a user scrolls through indications by a wheel on an input device such as a mouse or a keyboard. Alternatively, a user may scroll by moving a mouse cursor in a lateral and/or longitudinal motion. Furthermore, scrolling may be performed by selecting predetermined keys on a keyboard such as, for example, arrow keys. On another example implementation, a user may scroll by using a PACS workstation 140 with a touchscreen monitor and moving an input device (i.e., a touchscreen pen or a finger) across the touchscreen. The scroll cine manager 150 includes configuration such that a user may select their preferred method of scrolling. A user may scroll through the images in a numerically ascending order and/or in reverse order depending on the direction of the scrolling movement.

The example scroll cine manager 150 provides one or more indications as to the location of images within the stack. A user of the scroll cine manager 150 scrolls through the images in the stack with the scroll cine manager 150 displaying an indication of which image the user has scrolled to without requiring the user to select the indication to view the image. In an example implementation, the indication is a thumbnail image representative of the corresponding larger full resolution image. The thumbnail image enables a user to preview an image before selecting the image for display in the user interface. By previewing an image, a user can quickly determine if the displayed thumbnail image is the desired image to display. If the image is not the correct image the user simply continues scrolling until the desired image is displayed. The scroll cine manager 150 tracks the scrolling by a user such that the thumbnail images and/or other indications are displayed as the user is scrolling. For example, if a user quickly scrolls, the scroll cine manager 150 changes the thumbnail image displayed and/or other indication information at the rate the user is scrolling. Additionally, the scroll cine manager 150 may skip one or more thumbnail images and corresponding indication information if a user scrolls at a very high rate.

The indication includes a scale representing image location within the stack. The scale includes tick marks, arrows, or any other type of visual indicator positioned within the scale according to the scrolled location of the indication within the stack. For example, if a stack includes 250 images, the scale would show lines representing different points in the stack. A line at the midpoint of the scale represents the 125^{th} image within the stack. If a user scrolls to the 200^{th} image in the stack, the tick mark or arrow is positioned at the 80% mark on the scale. A user may scroll through the images by selecting the tick mark and/or arrow and moving the tick mark or arrow along the scale to the desired image. Alternatively, the scroll cine manager 150 may display the tick marks and/or arrows without the scale within the user interface. The position of the tick marks or arrows within the user face indicates the relative scroll indication of the image location in the stack. For example, the tick marks may be displayed on a perimeter of the user interface or adjacent to the image displayed within the user interface.

Additionally, the indication generated by the example scroll cine manager 150 may include text information describing a name assigned to the image, a frame number of the image, a numerical reference of the image, a slice location of the image, and/or a series number of the image, for example. This text information in the indication provides a user with information regarding the image location within the stack. The name assigned to an image may be assigned by a hanging protocol, a display protocol, the imaging modality 110 that generated the image, the acquisition workstation 120 that processed the image, the PACS server 130, and/or by a user of a PACS workstation 140, for example. The name may include a description of the image, a note provided by a user viewing the image, or a flag indicating a significant and/or a key image for making a diagnosis, for example.

The scroll cine manager 150 of FIG. 1 enables a user to select an indication of an image. Upon selecting a displayed indication the scroll cine manager 150 displays the image corresponding to the selected indication. At this point, the scroll cine manager 150 enables the user to continue scrolling through indications of images. Alternatively, as a user scrolls through the index referencing the stack the scroll cine manager 150 displays the corresponding medical image in the user interface. In this implementation, a user can select the displayed image to maintain the display of the image or deselect the image and return to the image displayed prior to scrolling. Furthermore, while using the scroll cine manager 150 a user may invoke other features, applications and/or functionalities of the user interface to examine the image.

In another example implementation, the scroll cine manager 150 provides one or more indications as to the location of slice images within a primary image. The primary image may be a three-dimensional image or a two-dimensional image with the slice images in the image stack corresponding to locations within the primary image. For example, a primary three-dimensional image of a brain includes cross sectional image slices. A user may select a location on the three-dimensional image to display a cross section slice image of the selected location. The scroll cine manager 150 displays the three-dimensional image and an indication located in the selected part of the three-dimensional image corresponding to the displayed slice image.

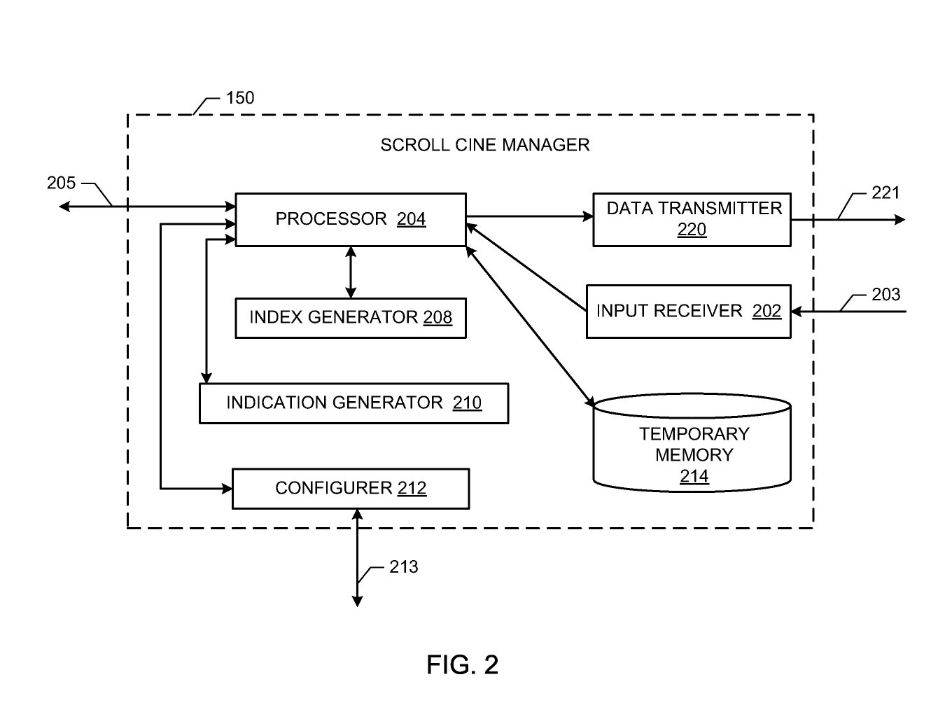

FIG. 2 shows a functional diagram of the example scroll cine manager 150 of FIG. 1 including an input receiver 202, a processor 204, an index generator 208, an indication generator 210, a configurer 212, and a data transmitter 220. Additionally, the scroll cine manager 150 includes connections 203, 205, 213, and 221 to the PACS server 130. The connections 203, 205, 213, and 221 may include any types of electrical and/or mechanical connections to enable communication between the scroll cine manager 150 and the PACS server 130. The connections 203, 205, 213, and 221 may go through the respective PACS workstation 140 or alternatively, the connections 203, 205, 213, and 221 may directly connect to the PACS server 130. The scroll cine manager 150 receives instructions and data from the PACS server 130 via the connection 205 connected to the processor 204. The instructions may include activation instructions that a PACS workstation 140 has opened a user interface for examining a stack of images or deactivation instructions indicating the user has closed the user interface and/or deactivated the image scrolling application within the user interface. The instructions from the PACS server 130 may provide the scroll cine manager 150 with the network address of the PACS workstation 140 viewing an image stack and/or an image stack location in the memory of the PACS server 130.

In addition to any number and/or type(s) of specialized hardware, firmware and/or logic to perform processing functions, the example processor 204 of FIG. 2 includes any number and/or type(s) of specialized and/or general purpose controller(s) and/or processing unit(s) capable of executing coded instructions. For example, the controller and/or processing unit may perform any number and/or type(s) of processing functions by carrying out and/or executing coded instructions present in a memory communicatively coupled and/or within the processor 204 (e.g., within a random-access memory (RAM), a read-only memory (ROM) and/or on-board memory of the processor 204).

The processor 204 manages the functions of the example scroll cine manager 150. The functions including processing inputs generated by users of the PACS workstations 140, loading an image stack from the PACS server 130, saving an image stack to a temporary memory 214, coordinating the creation of an index and indications corresponding to an image stack, tracking user scrolling inputs to indications on the an index, transmitting the indications to be displayed within the user interface on the PACS workstations 140, transmitting a selected image for display within the user interface, and/or communicating configuration information to the PACS server 130. In an alternative implementation, the processor 204 may process an image stack in the memory of the PACS server 130 without loading the image stack into the temporary memory 214 of the scroll cine manager 150.

The temporary memory 214 may be any number and/or type(s) of memories and/or memory devices including RAM and/or ROM memory. The temporary memory 214 stores one or more image stacks including the images within the image stacks. The processor 204 accesses the temporary memory 214 for generating indexes and indications of image locations within the stack. Additionally, the processor 204 accesses the temporary memory 214 to display one or more images within the image stack in the user interface. The temporary memory 214 may retain the stack until a user ends an image viewing session on the PACS workstation 140 and/or may retain the stack until a user manually removes the stack from the user interface on the PACS workstation 140. Alternatively, the temporary memory 214 may store a stack until a specified time in which the scroll cine manager 150 deletes the stack from the temporary memory 214.

The index generator 208 of FIG. 2 creates an index from an image stack saved in the temporary memory 214. The index generator 208 receives the information regarding the image stack from the processor 204 and creates an index upon receiving a command from the processor 204. The index created by the index generator 208 links the location of the images within the stack stored in the temporary memory 214 to the indication information generated by the indication generator 210. Furthermore, the index generator 208 and/or the processor 204 may link the indication information to the index. The index generator 208 generates the index based on the order of the images within the stack. Additionally or alternatively, the index generator 208 creates the index from information associated with the images in the stack. This may include DICOM header information within the image and/or any other information within the image file created by a hanging protocol or a perspective.

The processor 204 uses the index to quickly track scrolling by a user enabling the display of the indication corresponding to the amount of scrolling. Additionally, the processor 204 uses the index to locate the image in the stack if a user selects the indication of the image for display in the user interface. For example, if the processor 204 receives scrolling information indicating a user is scrolling in a descending manner through indications at a rate of one indication per 100 milliseconds, the processor 204 moves down the index at the same rate, thus displaying the indications in the user interface as the processor 204 reaches the indication reference in the index.

The indication generator 210 creates indications of image location information within the image stack. The indication generator 210 receives the information regarding the image stack from the processor 204 and creates an index upon receiving a command from the processor 204. The indications generated by the indication generator 210 include thumbnail images of the corresponding images in the stack, text information including image location within the stack and/or descriptions of the images, and/or scales representing image locations within the stack. Additionally, the indication generator 210 may include an icon, tick mark, and/or arrow in the scale to mark the current displayed image and/or the scrolling location. The indication generator 210 generates indications for the images in the stack specified by the processor 204. This may include all of the images in the stack or alternatively only some of the images in the stack. Upon creating the indications, the indication generator 210 transmits the indications to the processor 204 for linking with the index. By creating the indications prior to a user scrolling through the indications, the indications are quickly displayed within the user interface enabling faster scrolling and accurate tracking of indication display with user inputs.

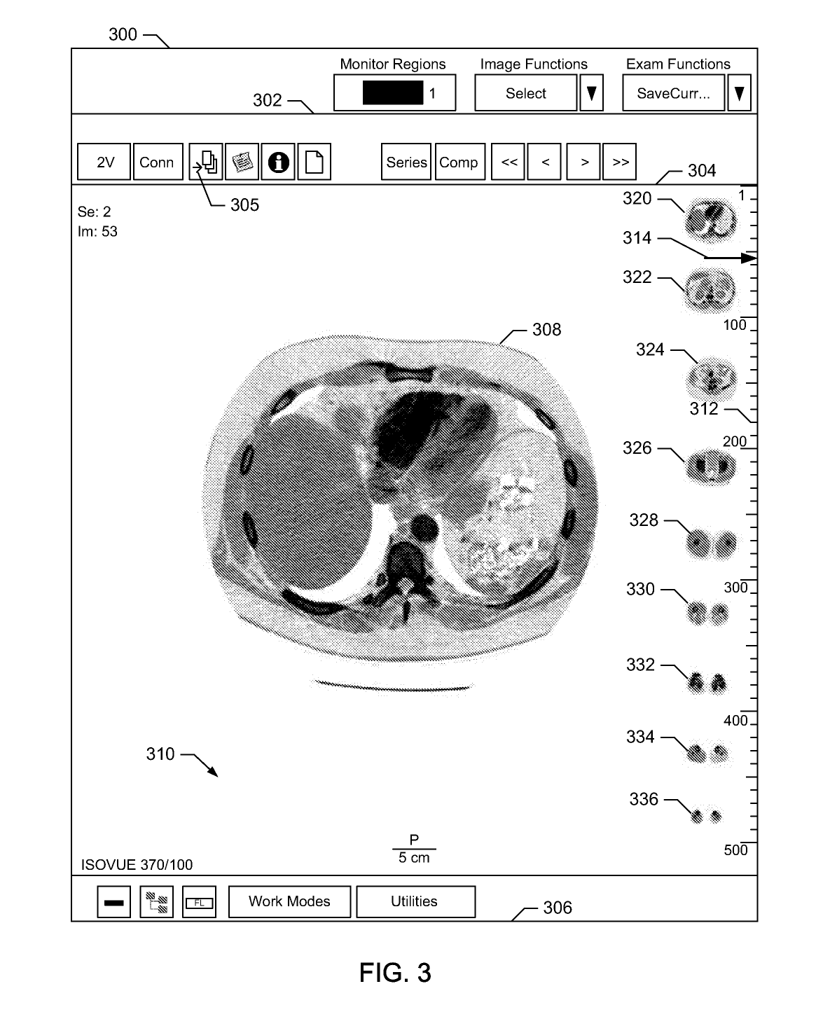

The processor 204 displays the scale and image location within the user interface upon a user invoking the image scrolling tool in the user interface. In some example implementations, the processor 204 may display thumbnail images adjacent to the scale to representing they type of information displayed on images in that part of a scale. An example is described in further detail in connection with FIG. 3.

The indication generator 210 creates thumbnail images from the images in the stack by scaling the images in the stack into the size of a thumbnail display and reducing the resolution of the thumbnail image. Furthermore the indication generator 210 creates text indication information by referencing the image location within the stack. Additionally or alternatively, the indication generator 210 creates the indications from information associated with the images in the stack. This may include DICOM header information within the image, user generated information saved within the image file or attached to the image file and/or any other information within the image file created by a hanging protocol or a perspective. For example, an image in a stack is the third image in the tenth series in the stack. Accordingly, the indication created by the indication generator 210 would be “Se: 10 Im: 3 SL: 58” (“Se” is the series number, “Im” is the image stack number in the series, and “SL” is the slice number in the stack). In the example indication, the SL: 58 indicates the image is the 58^{th} image in the stack. The series number may reference a group of similar images (e.g., the outer layer of the lungs of a patient X) generated by the imaging modality 110.

The scroll cine manager 150 of FIG. 1 includes an example input receiver 202 to receive user generated inputs on the PACS workstations 140. The inputs may pass from the PACS workstations 140 to the PACS server 130 which then forwards the inputs via the connection 203 to the input receiver 202. Upon receiving the input, the input receiver 202 forwards the input to the processor 204. Alternatively, the input receiver 202 may receive inputs directly from the PACS workstations 140. The inputs received by the input receiver 202 include scrolling by a mouse cursor or a keyboard entry, selections of indications, selections or specifications of scroll cine manager 150 configuration information, and/or any other type of image viewing inputs. For example, if a user scrolls through a stack of images using the wheel of a mouse, the input receiver 203 receives the digital data indicating the direction of the movement of the wheel. The input receiver 202 forwards this movement information to the processor 204 so that the processor 204 can accumulate the number of movements by the wheel to display the corresponding indications.

In the example implementation of FIG. 2, the scroll cine manager 150 includes the data transmitter 220 to forward indications of image locations and images to the PACS workstations 140 for display. The data transmitter 220 receives the information to forward from the processor 204. Upon receiving information from the processor 204, the data transmitter 220 forwards the information via the connection 221 to the respective PACS workstation 140. Alternatively, the data transmitter 220 may forward the information directly to the PACS server 130 for display on another PACS workstation 140. The data transmitter 220 ensures the PACS workstation 140 receives the information. In cases where the information is not received, the data transmitter 220 may continue to resend the information until the information is received by the PACS workstation 140. Additionally, the data transmitter 220 may send control level information for establishing a link to a PACS workstation 140.

For example, if a PACS workstation 140 invokes a user interface with image scrolling, the processor 204 receives the network address of the PACS workstation 140 from the PACS server 130. The processor 204 may send one or more control messages to the PACS workstation 140 via the data transmitter 220 to establish a communication link such that the processor 204 may subsequently send indication information and/or images from the stack to the same workstation 140. In other example implementations, the data transmitter 220 may be responsible for sending indication information and/or images from different stacks to the corresponding PACS workstations 140. In this case, the data transmitter 220 uses stack information included in the indication information and/or the image to send the information to the appropriate PACS workstation 140.

The scroll cine manager 150 includes a configurer 212 to enable users of the scroll cine manager 150 to specify criteria and/or configurations for displaying indications in the user interface. The configurer 212 connects to the PACS server 130 via the connection 213 for communicating display configuration information. The configurer 212 may interface with hanging protocols and/or perspectives within the PACS server 130 for displaying images and indications in the user interface. Alternatively, the configurer 212 may be directly coupled to the PACS workstations 140 and store user specific scroll cine manager 150 configuration information in the temporary memory 214.

The configurer 212 may be integrated with the PACS server 130 such that a list of configuration options is stored within the configurer 212 and the options selected for certain users and/or user interfaces is stored in the PACS server 130. A user accesses the configurer 212 through icons within the user interface such as, for example, a utilities icon that opens into a list of display options.

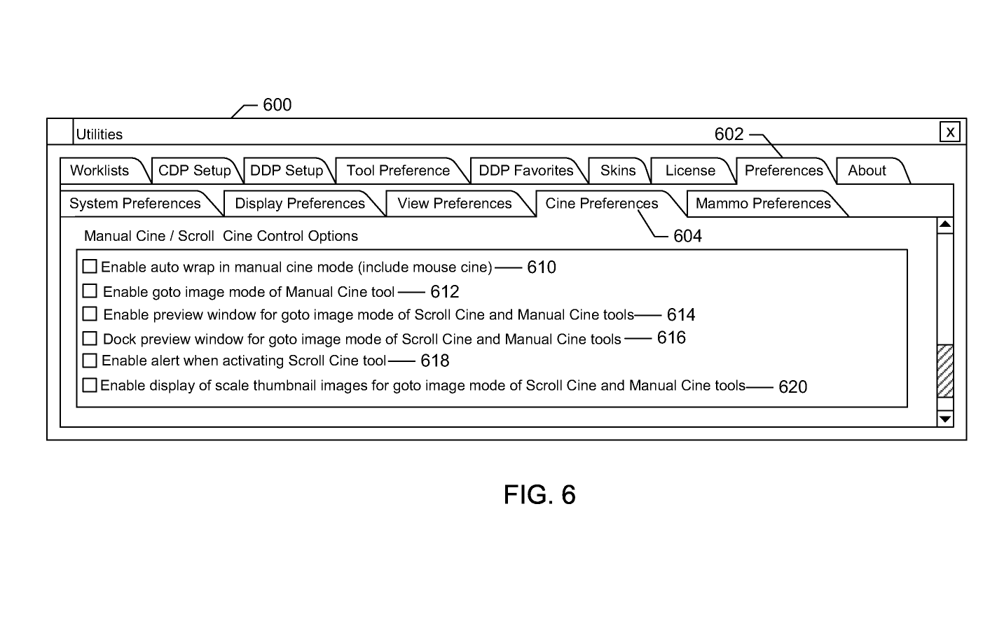

The configurer 212 enables a user to specify the type of indication for display in the user interface. This includes configuring the user interface for displaying representative thumbnail images adjacent to a scale corresponding to the image stack, displaying a scale with a tick marker and/or arrow representing the scroll image location in the stack, displaying text information describing an image position in the stack and/or information about the image, and/or displaying a thumbnail image of the corresponding image in the stack. Additionally, the configurer 212 enables a user to specify if the scroll cine manager 150 is activated upon opening a user interface or by a user selecting an icon in the user interface. A further description of configuration options is described in connection with FIG. 6.

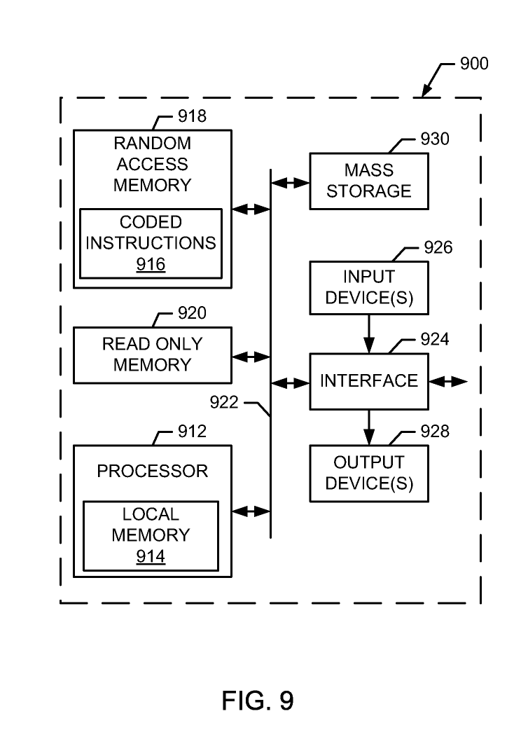

While an example manner of implementing the scroll cine manager 150 of FIG. 1 has been illustrated in FIG. 2, one or more of the interfaces, data structures, elements, processes and/or devices illustrated in FIG. 2 may be combined, divided, rearranged, omitted, eliminated and/or implemented in any other way. For example, the example input receiver 202, the example processor 204, the example index generator 208, the example indication generator 210, the example configurer 212, and/or the example data transmitter 220 illustrated in FIG. 2 may be implemented separately and/or in any combination using, for example, machine accessible instructions executed by one or more computing devices and/or computing platforms (e.g., the example processing platform 900 of FIG. 9). Further, the example input receiver 202, the example processor 204, the example index generator 208, the example indication generator 210, the example configurer 212, the example data transmitter 220, and/or, more generally, the scroll cine manager 150 may be implemented by hardware, software, firmware and/or any combination of hardware, software and/or firmware. Thus, for example, any of the example input receiver 202, the example processor 204, the example index generator 208, the example indication generator 210, the example configurer 212, the example data transmitter 220, and/or, more generally, the scroll cine manager 150 could be implemented by one or more circuit(s), programmable processor(s), application specific integrated circuit(s) (ASIC(s)), programmable logic device(s) (PLD(s)) and/or field programmable logic device(s) (FPLD(s)), etc. When any of the appended claims are read to cover a purely software or firmware implementation, at least one of the example input receiver 202, the example processor 204, the example index generator 208, the example indication generator 210, the example configurer 212, the example data transmitter 220, and/or, the scroll cine manager 150 are hereby expressly defined to include a tangible medium such as a memory, DVD, CD, etc. storing such software or firmware. Further still, the example scroll cine manager 150 may include additional devices, servers, systems, networks, gateways, portals, and/or processors in addition to, or instead of, those illustrated in FIG. 2 and/or may include more than one of any or all of the illustrated devices, servers, networks, systems, gateways, portals, and/or processors.

FIG. 3 shows an example user interface 300 displaying indications of image location in a stack managed by the scroll cine manager 150 of FIG. 2. The user interface 300 includes tool bars 302, 304, and 306 which provide access to configuration menus, navigation tools, viewing tools, imaging applications, file management tools, etc. The tool bars 302, 304, and 306 may include additional icons and/or functionalities or less icons and/or functionalities shown. A scroll cine manager icon 305 is shown within the navigation tool bar 304. The scroll cine manager icon 305 activates the functionalities of the scroll cine manager 150 upon a user selecting the scroll cine manager icon 305. Additionally, by selecting the scroll cine manager icon 305 a second time the scroll cine manager 150 and its associated functionalities may be deactivated. In other examples, the scroll cine manager icon 305 may be included in a tool palette that contains icons for image modification, image navigation, and/or image processing functions. The tool palette may be selectable via an icon in any one of the tool bars 302, 304 and/or 306.

The user interface 300 includes a display area 310 for displaying a medical image 308. Additionally, the display area 310 displays associated information regarding the medical image 308 including any information included in a file of the medical image 308 generated by the imaging modality 110 and/or the acquisition workstation. This information may include the type of imaging modality 110 used to generate the image, patient identification information, image creation date and time, patient medical information, etc. In the example implementation of FIG. 3, the medical image 308 is part of an image stack temporarily stored in the temporary memory 214 of FIG. 2.

The user interface 300 of FIG. 3 shows an indication of image location via a scale 312 representing the images in the stack. In the example of FIG. 3, the stack includes 500 medical images represented by the scale ranging from 1 to 500. An arrow 314 is used to point to the location in the scale 312 if the currently displayed medical image 308 (i.e., image 53). Adjacent to the scale 312 are thumbnail images 320-336. The thumbnail images 320-336 provide representative indication information regarding the types of images located in each part of the scale 312. For example, the thumbnail image 320 shows a slice image of an exterior set of lungs. This indication informs a user that images similar to the thumbnail image 320 are located in this part of the scale 312. The thumbnail image 334 shows a slice image of a focused part interior to the same lungs. This indication informs the user that images similar to the thumbnail image 334 are located in this part of the scale 312. By knowing the relative position of representative images these thumbnail images 320-336 enable a user to quickly scroll through the scale 312 to find a desired image.

A user may scroll through the images by selecting the arrow 314 and moving the arrow along the scale 312. Additionally, the user may move a mouse cursor or select keys of a keyboard. The arrow 314 would track of the key presses and/or mouse cursor movements by the user. Alternatively, a user may user a touchscreen pen or finger to select the arrow 314 and move it along the scale 312 and/or the user may move his or her finger in a scrolling position on any location within the display area 310. Furthermore, a user may scroll through the images in a numerically ascending order and/or in reverse order based on the direction of the scrolling movement. Depending on the configuration settings, the medical image in the display area 310 may change to the corresponding image as the user scrolls through the scale 312 or the medical image 308 is displayed until the user selects another image by moving the arrow 312 to another number in the scale 312 and selects the indication at that number.

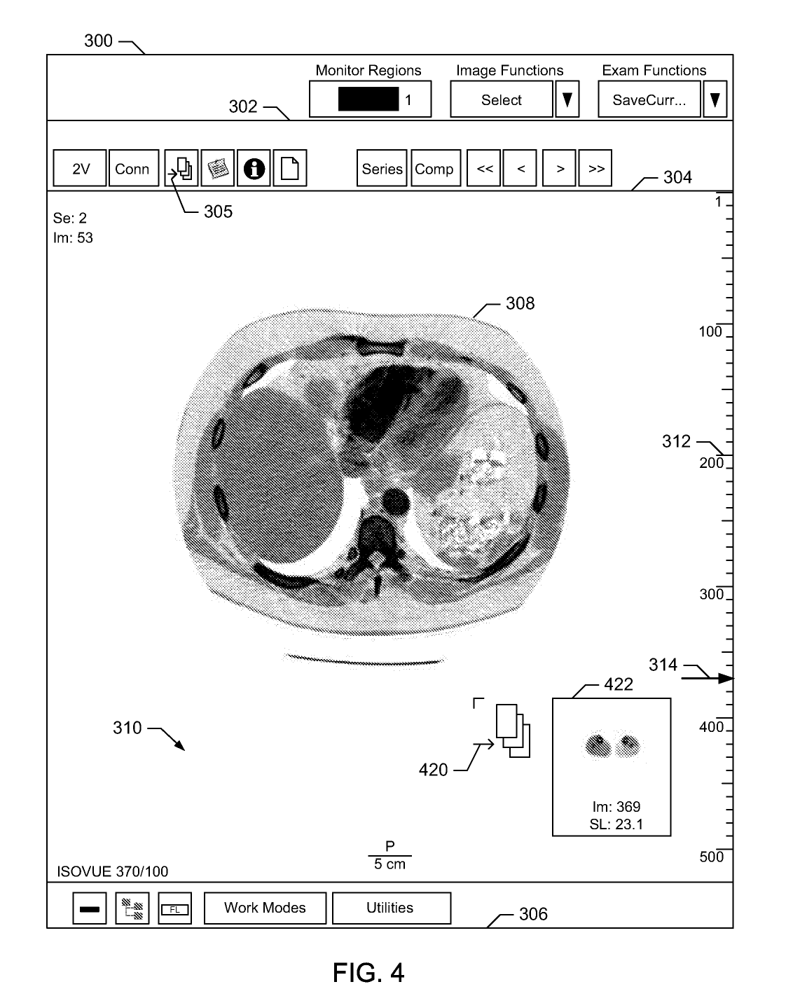

FIG. 4 shows the example user interface 300 of FIG. 3 displaying an indication of an image location in a stack managed by the scroll cine manager 150 of FIG. 2. The user interface 300 includes the tool bars 302, 304, and 306, the scroll cine manager icon 305, the display area 310, the scale 312, the arrow 314 and the medical image 308 of FIG. 3. Additionally, the user interface 300 includes a scroll cine icon 420 and a preview box 422. The scroll cine icon 420 is displayed upon activation of the scroll cine manager 150 and indicates to a user that the scrolling function is active in the user interface. The preview box 422 includes an indication of an image location in the stack by displaying a thumbnail image and text describing the image location in the stack. In other example implementations, the preview box 422 may contain just the thumbnail image or just the text indication.

A user may scroll through images in the stack by scrolling in the preview box 422. The preview box 422 provides feedback to a user indicting which image the user interface will display upon selecting the displayed indication. This enables quick location of desired images in an image stack. For example, a user quickly scrolls to a desired image by moving the mouse wheel causing the thumbnail image and text information in the preview box 422 to track the wheel movement. FIG. 4 shows that the medical image 308 (image 53) is displayed in the display area 310 and the preview box 422 displays a thumbnail image corresponding to image 369 in the stack. A user may select the thumbnail image in the preview box 422 resulting in the display of image 369 in the display area 310. Alternatively, a user may continue to scroll in the preview box 422 until the desired image is located. Additionally, the arrow 314 moves in the scale 312 pointing to the image location in the stack as the user scrolls in the preview box 422. Furthermore, a user may scroll by moving the arrow 314 along the scale 312 to the desired image. As the user moves the arrow 314 the indication in the preview box 422 displays the corresponding thumbnail image and text location.

In an alternative implementation, the preview box 422 may include a thumbnail image of a primary image and an indication of the location of a selected cross sectional slice image in the primary image. The primary image may be a three-dimensional image or a two-dimensional image of a structure with image slices displaying cross sectional views of the structure. A user may select a location on the primary image or may scroll via the arrow 314 to select and display additional cross sectional images of the primary image.

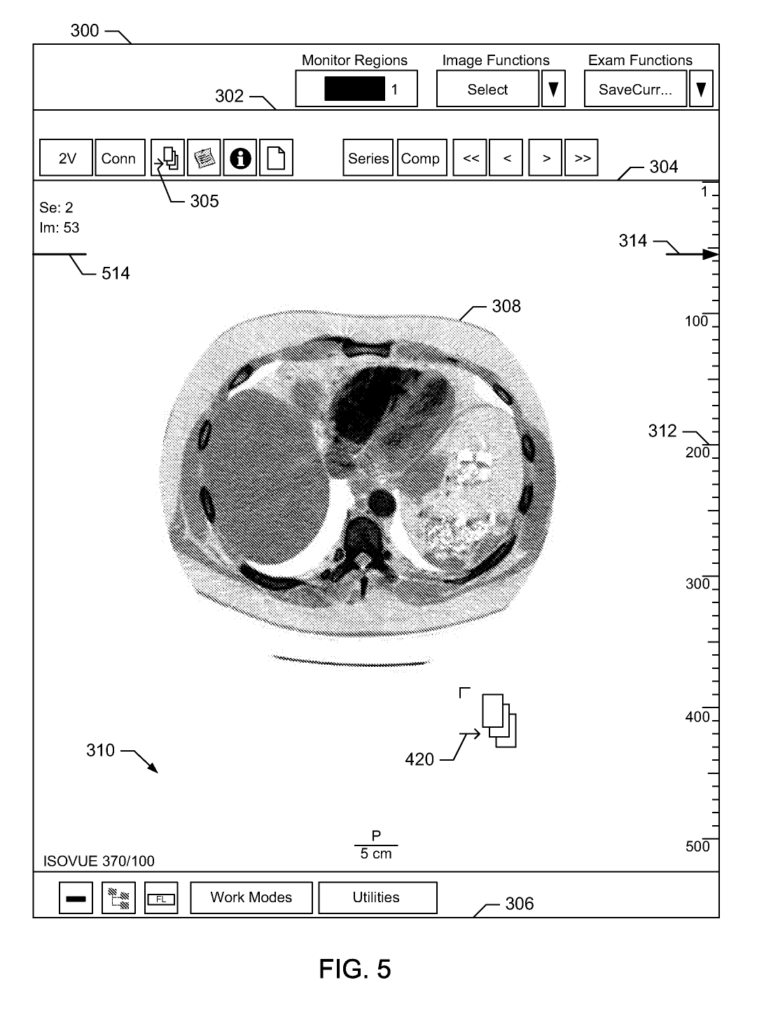

FIG. 5 shows the example user interface 300 of FIG. 3 displaying an indication of an image location in a stack managed by the scroll cine manager 150 of FIG. 2. The user interface 300 includes the tool bars 302, 304, and 306, the scroll cine manager icon 305, the display area 310, the scale 312, the arrow 314 and the medical image 308 of FIG. 3 and the scroll cine icon 420 of FIG. 4. The scroll cine icon 420 indicates the scrolling cine function is active enabling a user to scroll through medical images. Additionally, the user interface 300 includes a tick mark 514 indicating the location of the medical image 308 in the image stack. Like the arrow 314 in the scale 312, the tick mark 514 indicates the relative position of the medical image 308.

A user may select the tick mark 514 and move the tick mark 514 in the display area 310 causing the user interface 300 to scroll through images in the stack. Scrolling may also be performed by moving the mouse cursor and/or the mouse wheel. In this case the tick mark 514 and arrow 314 track the mouse cursor movement and display the scrolled location in the stack. As the tick mark 514 is moved, the corresponding medical image may be displayed in the display area 310. Alternatively, the scrolled to image may not be displayed in the display area 310 until the user selects the tick mark 514 at the point the scrolling is stopped. Furthermore, if a user quickly scrolls through the images, the display area 310 may skip some images to display. In this case a warning indicator may be displayed in the display area 310 indicating images have been skipped.

In an alternative implementation, the example scroll cine manager 150 may manage images in a sheet mode for display in the display area 310. Images in a sheet mode may include a row of images and/or a sheet of images displayed on a single strip of film and/or a film sheet. The scroll cine manager 150 organizes the images in the sheet mode in the same manner as images in a stack are managed. Likewise, a user may scroll through the images in the sheet mode the same way a user would scroll through images in the image stack. Furthermore, the image preview may be displayed as a thumbnail image and/or as a text indicator of the image within a sheet of images.

FIG. 6 shows an example configuration interface 600 for configuring and selecting preferences for the scroll cine manager 150. The configuration interface 600 is accessed by selecting a Utilities icon in the tool bar 306 of FIG. 3. Within the configuration interface 600, a user selects the Preferences tab 602 and then the Cine Preferences tab 604 to view the scroll cine manager 150 configuration options. The options shown in FIG. 6 are example configuration options and are in no way limiting to the types of options a user and/or a system developer may configure for the scroll cine manager 150.

The configuration options include an ‘Enable auto wrap in manual mode’ option 610 for enabling the scroll cine manager 150 to scroll from a last image in a stack to the first image in the stack or vice versa. The ‘Enable goto image mode of Manual Cine tool’ option 612 enables a user to select and image once the user scrolls to the indication. The ‘Enable preview window for goto image mode of Scroll Cine and Manual Cine tools’ option 614 activates the preview box 422 when the scrolling functionality of the user interface is active. The ‘Dock preview window for goto image mode of Scroll Cine and Manual Cine tools’ option 616 sets the preview box 422 adjacent to the image in the display area. Otherwise, if the option 616 is not selected the preview box 422 tracks the movement of the mouse cursor. The ‘Enable alert when activating Scroll Cine tool’ option 818 enables a message to be displayed on the user interface when the scrolling cine tool is activated by selecting an icon in the tool bar or by pressing a hotkey on a keyboard. The ‘Enable display of scale thumbnail images for goto image mode of Scroll Cine and Manual Cine tools’ option 620 enables the display of representative thumbnail images adjacent to a scale in the display area 310 similar to the thumbnail images 320-336 of FIG. 3. In other example implementations, the configuration interface 600 may include options for selecting the text type displayed for indicating image location in the stack, maximum scroll speed, method of scrolling (e.g., mouse cursor, mouse wheel, keyboard keys, touchscreen, etc), etc.

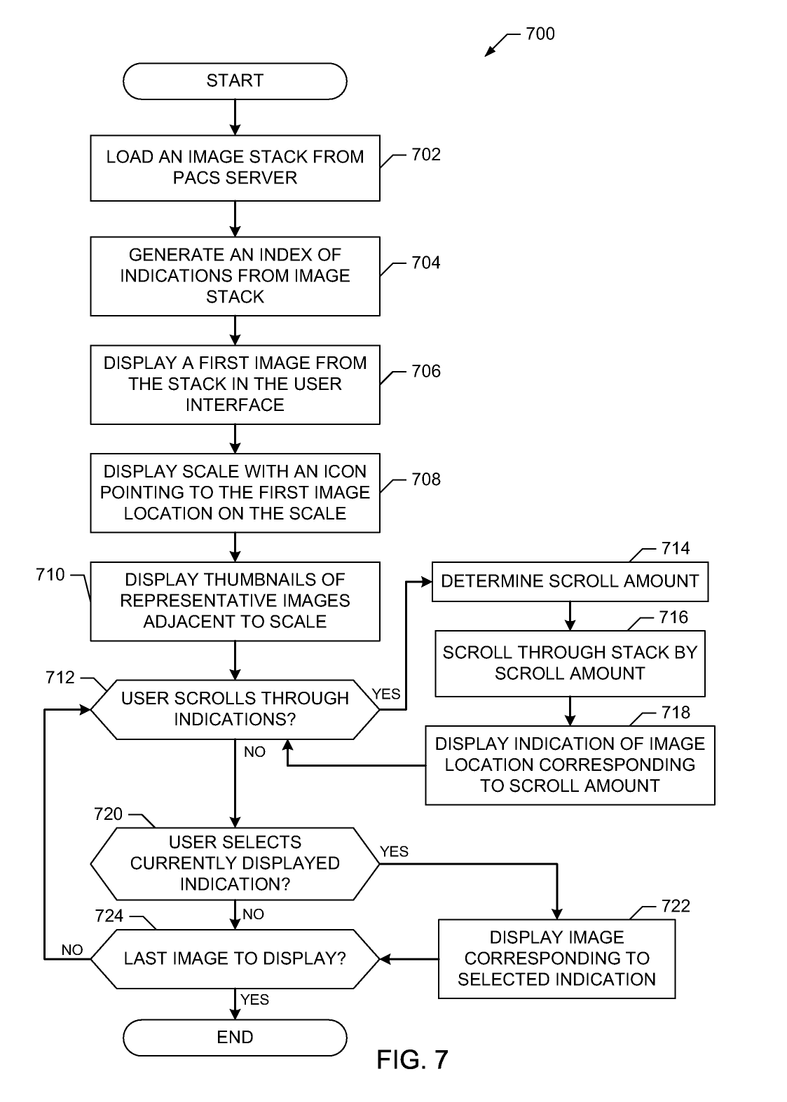

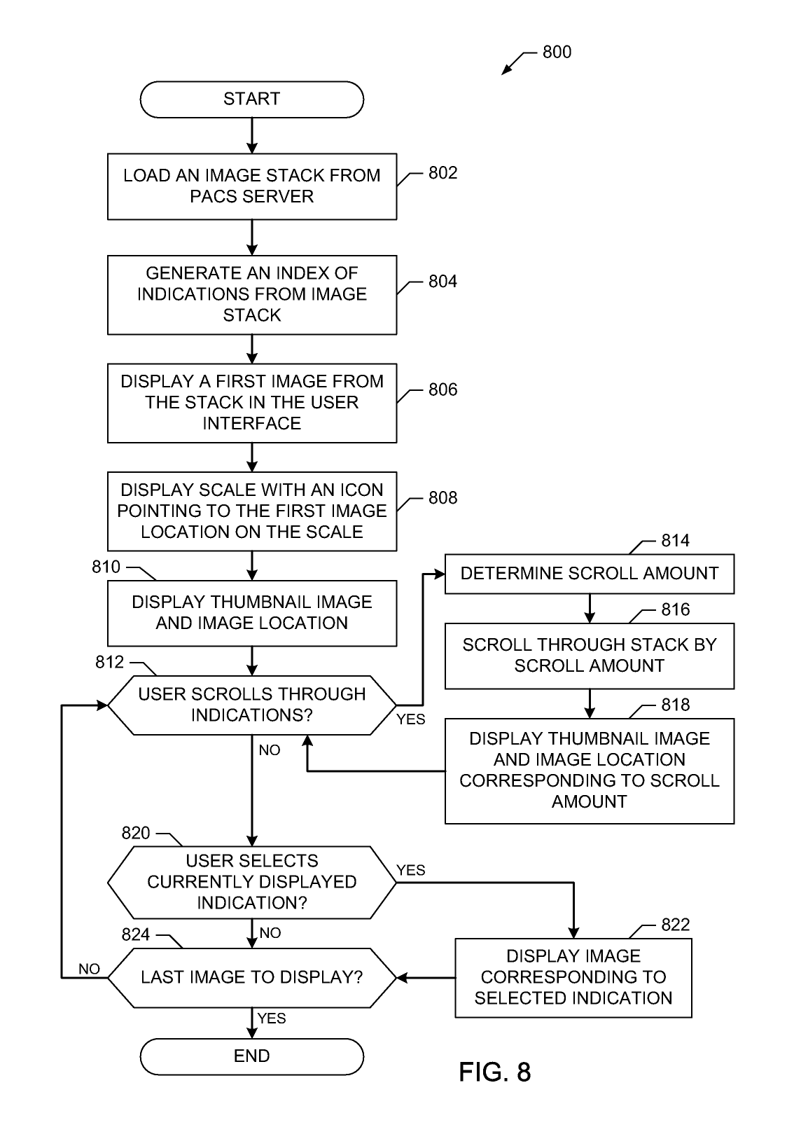

FIGS. 7 and 8 are flowcharts representative of example machine readable instructions that may be executed to indicate image location in an image stack in the scroll cine manager 150 of FIGS. 1 and 2. The example machine readable instructions may be executed using, for example, a processor system such as the system 900 of FIG. 9. However, one or more of the blocks depicted in the flowcharts may be implemented in any other manner, including by dedicated purpose circuitry, manual operations, etc. Additionally, although the example instructions are described with reference to the flowcharts of FIGS. 7 and 8, other methods to indicate image location in an image stack in the scroll cine manager 150 may additionally or alternatively be used. For example, the order of execution of the blocks depicted in the flowcharts of FIGS. 7 and/or 8 may be changed, and/or some of the blocks described may be rearranged, eliminated, or combined.

The example instructions 700 represented by FIG. 7 may be performed to implement any or all of the example scroll cine manager 150 of FIGS. 1 and/or 2. The example instructions 700 may be executed at predetermined intervals (e.g., hourly, yearly, etc), in response to an occurrence of a predetermined event or trigger (e.g., the scroll cine manager 150 being activated upon opening a user interface), in response to a user request, or on any combination thereof.

The example instructions 700 of FIG. 7 begin when the scroll cine manager 150 loads an image stack from the PACS server 130 of FIG. 1 (block 702). The scroll cine manager 150 may load the image stack in response to the opening of a user interface and/or in response to a user activating the scroll cine manager 150 and selecting an image stack to view from the user interface. Upon loading the image stack, the scroll cine manager 150 generates an index of indications from the image stack (block 704). The generated indications may include thumbnail images of corresponding images in the stack, text information describing the corresponding location of an image in the stack, a scale to represent the image stack, and/or icons or arrows for display within the scale.

The example instructions 700 continue when the scroll cine manager 150 displays the first image in the stack in the user interface (block 706). Alternatively, the scroll cine manager 150 may display the last image viewed in the stack from a previous session. Next, the scroll cine manager 150 displays the scale with an icon pointing to the location on the scale of the displayed image, representing the location of the image in the stack (block 708). Then, the scroll cine manager 150 displays thumbnail images of representative images in the stack adjacent to the scale (block 710). Upon displaying the indications and the image in the user interface, the scroll cine manager 150 determines when a user scrolls through the images (block 712). If the user does not scroll, the scroll cine manager 150 determines if the user selects the indication displayed (block 720). If the user has not selected another indication the image remains displayed in the user interface and the scroll cine manager 150 determines if the user closes the user interface or if the displayed image is the final image the user intends to scroll to (block 724). If the user intends to continue to use the scroll function, control returns to block 712 and the scroll cine manager 150 determines if the user is initiating scrolling.

If in the example instructions 700 the user scrolls through the indications, the scroll cine manager 150 determines the amount of scrolling (block 714). The amount of scrolling may include the scrolling rate and/or the number of indications scrolled through. Upon determining the scroll amount, the scroll cine manager 150 scrolls through the index by the scroll amount (block 716) and displays the indications of the image location in the scale as it scrolls through the indications in the index (block 718). Next, the scroll cine manager 150 determines if the user is still scrolling (block 712). If the user continues to scroll, control loops through blocks 714-718 until the user stops scrolling. Upon the scroll cine manager 150 determining the scrolling has stopped, the scroll cine manager 150 determines if the user selects the currently displayed indication (720). If the scroll cine manager 150 receives an input indicating the user selects the indication, the scroll cine manager 150 displays the image in the user interface corresponding to the selected indication (722). Next, the scroll cine manager 150 determines if the user closes the user interface or if the displayed image is the final image the user intends to scroll to (block 724). If the user intends to continue to use the scroll function, control returns to block 712 and the scroll cine manager 150 determines if the user initiates scrolling. If the scroll cine manager 150 receives an input indicating the user has terminated the scrolling functionality in the user interface and/or closes the user interface, the example instructions 700 end.

The example instructions 800 represented by FIG. 8 may be performed to implement any or all of the example scroll cine manager 150 of FIGS. 1 and/or 2. The example instructions 800 may be executed at predetermined intervals (e.g., hourly, yearly, etc), in response to an occurrence of a predetermined event or trigger (e.g., the scroll cine manager 150 being activated upon opening a user interface), in response to a user request, or on any combination thereof.

The example instructions 800 of FIG. 8 begin when the scroll cine manager 150 loads an image stack from the PACS server 130 of FIG. 1 (block 802). Similar to the example instructions 700 in FIG. 7, the example instructions in FIG. 8 generate an index of indications from the image stack (block 804), display a first image from the stack in the user interface (block 806), and display a scale with an icon pointing to the location of the first image in the stack (block 808). Next, the scroll cine manager 150 displays a thumbnail image and text describing the location of the displayed image in the user interface (block 810). The thumbnail image and text may be displayed within a preview box adjacent to a displayed image in the user interface.

Next, the scroll cine manager 150 determines if the user scrolls through the indications (block 812). A user may scroll the indications in the preview box and/or by moving the arrow in the scale. If in the example instructions 800 the user scrolls through the indications, the scroll cine manager 150 determines the amount of scrolling (block 814). Upon determining the scroll amount, the scroll cine manager 150 scrolls through the index by the scroll amount (block 816) and displays the thumbnail image and text information for each image as it scrolls through the index (block 818). Next, the scroll cine manager 150 determines if the user is still scrolling (block 812). If the user continues to scroll, control loops through blocks 814-818 until the user stops scrolling.

Upon the scroll cine manager 150 determining the scrolling has stopped, the scroll cine manager 150 determines if the user selects the currently displayed thumbnail in the preview box (820). If the scroll cine manager 150 receives an input indicating the user selects the indication, the scroll cine manager 150 displays the image in the user interface corresponding to the selected thumbnail image (822). Next, the scroll cine manager 150 determines if the user closes the user interface or if the displayed image is the final image the user intends to scroll to (block 824). If the user intends to continue to use the scroll function, control returns to block 812 and the scroll cine manager 150 determines if the user initiates scrolling. If the scroll cine manager 150 receives an input indicating the user has terminated the scrolling functionality in the user interface and/or closes the user interface, the example instructions 800 end.

FIG. 9 is a block diagram of an example computer system 900 capable of implementing the systems and methods disclosed herein. The computer 900 can be, for example, a server, a personal computer, an internet appliance, or any other type of computing device. Any or all of the example scroll cine manager 150 of FIGS. 1 and/or 2 may be implemented by the example computer 900.

The system 900 of the illustrated example includes a processor 912 such as a general purpose programmable processor. The processor 912 includes a local memory 914, and executes coded instructions 916 present in the local memory 914 and/or in another memory device. The coded instructions 916 may include some or all of the instructions represented in FIGS. 7 or 8. The processor 912 may be any type of processing unit, such as one or more microprocessors from the Intel® Centrino® family of microprocessors, the Intel® Pentium® family of microprocessors, the Intel® Itanium® family of microprocessors, the Intel® Core® family of microprocessors, and/or the Intel® XScale® family of processors. Of course, other processors from other families are also appropriate.

The processor 912 is in communication with a main memory including a volatile memory 918 and a non-volatile memory 920 via a bus 922. The volatile memory 918 may he implemented by Static Random Access Memory (SRAM), Synchronous Dynamic Random Access Memory (SDRAM), Dynamic Random Access Memory (DRAM), RAMBUS Dynamic Random Access Memory (RDRAM) and/or any other type of random access memory device. The non-volatile memory 920 may be implemented by flash memory and/or any other desired type of memory device. Access to the main memory 918, 920 is typically controlled by a memory controller.

The computer 900 also includes an interface circuit 924. The interface circuit 924 may be implemented by any type of interface standard, such as an Ethernet interface, a universal serial bus (USB), and/or a third generation input/output (3GIO) interface.

One or more input devices 926 are connected to the interface circuit 924. The input device(s) 926 permit a user to enter data and commands into the processor 912. The input device(s) can be implemented by, for example, a keyboard, a mouse, a touchscreen, a track-pad, a trackball, an isopoint, and/or a voice recognition system.

One or more output devices 928 are also connected to the interface circuit 924. The output devices 928 can be implemented, for example, by display devices (e.g., a liquid crystal display, a cathode ray tube display (CRT)), by a printer and/or by speakers. The interface circuit 924, thus, typically includes a graphics driver card.

The interface circuit 924 also includes a communication device such as a modem or network interface card to facilitate exchange of data with external computers via a network (e.g., an Ethernet connection, a digital subscriber line (DSL), a telephone line, coaxial cable, a cellular telephone system, etc.).

The computer 900 also includes one or more mass storage devices 930 for storing software and data. Examples of such mass storage devices 930 include floppy disk drives, hard drive disks, compact disk drives and digital versatile disk (DVD) drives. The mass storage devices 930 may implement any or all of the example temporary memory 214. Additionally or alternatively, the volatile memory 918 may implement any or all of the example temporary memory 214.

At least some of the above described example methods and/or system are implemented by one or more software and/or firmware programs running on a computer processor. However, dedicated hardware implementations including, but not limited to, application specific integrated circuits, programmable logic arrays and other hardware devices can likewise be constructed to implement some or all of the example methods and/or apparatus described herein, either in whole or in part. Furthermore, alternative software implementations including, but not limited to, distributed processing or component/object distributed processing, parallel processing, or virtual machine processing can also be constructed to implement the example methods and/or apparatus described herein.

Certain example implementations contemplate methods, systems and computer program products on any machine-readable media to implement functionality described above. Certain example implementations may be implemented using an existing computer processor, or by a special purpose computer processor incorporated for this or another purpose or by a hardwired and/or firmware system, for example.

Certain example implementations include computer-readable media for carrying or having computer-executable instructions or data structures stored thereon. Such computer-readable media may be any available media that may be accessed by a general purpose or special purpose computer or other machine with a processor. By way of example, such computer-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of computer-executable instructions or data structures and which can be accessed by a general purpose or special purpose computer or other machine with a processor. Combinations of the above are also included within the scope of computer-readable media. Computer-executable instructions comprise, for example, instructions and data which cause a general purpose computer, special purpose computer, or special purpose processing machines to perform a certain function or group of functions.

Generally, computer-executable instructions include routines, programs, objects, components, data structures, etc., that perform particular tasks or implement particular abstract data types. Computer-executable instructions, associated data structures, and program modules represent examples of program code for executing steps of certain methods and systems disclosed herein. The particular sequence of such executable instructions or associated data structures represent examples of corresponding acts for implementing the functions described in such steps.

Embodiments of the present invention may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

To the extent the above specification describes example components and functions with reference to particular standards and protocols, it is understood that the scope of this patent is not limited to such standards and protocols. For instance, each of the standards for internet and other packet switched network transmission (e.g., Transmission Control Protocol (TCP)/Internet Protocol (IP), User Datagram Protocol (UDP)/IP, HyperText Markup Language (HTML), HyperText Transfer Protocol (HTTP)) represent examples of the current state of the art. Such standards are periodically superseded by faster or more efficient equivalents having the same general functionality. Accordingly, replacement standards and protocols having the same functions are equivalents which are contemplated by this patent and are intended to be included within the scope of the accompanying claims.

Additionally, although this patent discloses example systems including software or firmware executed on hardware, it should be noted that such systems are merely illustrative and should not be considered as limiting. For example, it is contemplated that any or all of these hardware and software components could be embodied exclusively in hardware, exclusively in software, exclusively in firmware or in some combination of hardware, firmware and/or software. Accordingly, while the above specification described example systems, methods and articles of manufacture, the examples are not the only way to implement such systems, methods and articles of manufacture. Therefore, although certain example methods, apparatus and articles of manufacture have been described herein, the scope of coverage of this patent is not limited thereto. On the contrary, this patent covers all methods, apparatus and articles of manufacture fairly falling within the scope of the appended claims either literally or under the doctrine of equivalents.

What is claimed is:

1. A method of indicating a medial image location in an image stack, the method comprising:
generating an index of indications of medical image locations in a medical image stack including a first indication of a first medical image location in a medical image stack and a second indication of a second medical image location in the medical image stack;
displaying in a user interface the first medical image and the first indication of the first medial image location within the medical image stack;
receiving a scroll input and scrolling through the index displaying indications of medical image locations in the image stack until the second indication of the second medical image is displayed;
receiving a selection of the second indication of the second medical image; and
displaying in the user interface the second medical image and the second indication of the second medical image location within the medical image stack.

2. A method as defined in claim 1, wherein the indications including the first indication and the second indication are at least one of a thumbnail image of the corresponding medical image, text describing the location of the corresponding medical image in the stack, an icon positioned within a scale, a thumbnail image of a primary image with a marker pointing to the location of the corresponding medical image in the stack, or an icon position adjacent to the corresponding medical image.

3. A method as defined in claim 2, wherein the scale corresponds to at least one of the index of indications or the medical image stack.

4. A method as defined in claim 2, wherein the icon position in the scale corresponds to the location of the associated indication in the medical image stack.

5. A method as defined in claim 4, further comprising displaying thumbnail images adjacent to the scale of corresponding medical images located at the corresponding location in the medical image stack.

6. A method as defined in claim 2, wherein the scale is a numerical scroll bar.

7. A method as defined in claim 2, wherein the icon position adjacent to the corresponding medical image corresponds to the location of the associated indication in the medical image stack.

8. A method as defined in claim 2, wherein text describing the location of the corresponding medical image in the stack includes at least one of a slice location of the medical image in the medical image stack, an image number of the medical image, a series number of the medical image, or the frame number of the medical image.

9. A method as defined in claim 1, further comprising upon generating the index, displaying in the user interface an icon indicating that the functionality of scrolling through the index is active.

10. A method as defined in claim 1, wherein the medical image stack is a group of medical images generated during one or more medical imaging procedures.

11. A method as defined in claim 1, wherein the indication of the medical image location is displayed at least one of adjacent to a medical image in the user interface or on top of a medical image in the user interface.

12. A method as defined in claim 1, wherein a scroll input includes at least one of moving a wheel on an input device, moving a mouse cursor in a lateral or longitudinal motion, selecting predetermined keys on an input device, or moving an input device on a touchscreen monitor displaying the user interface.

13. A scroll cine system to indicate a medial image location in an image stack, the system comprising:
an index generator to generate an index of indications of medical image locations in a medical image stack including a first indication of a first medical image location in a medical image stack and a second indication of a second medical image location in the medical image stack;
an input receiver to receive an amount of scrolling and to receive a selected indication; and
a processor to scroll through the index by the received amount of scrolling, to send to a user interface indications of medical image locations in the image stack as the indications are scrolled, and to send to the user interface a medical image corresponding to the received selected indication.

14. A scroll cine system as defined in claim 13, further comprising a display system including the user interface to display indications of medical image locations in the stack including the first indication and the second indication and displays medical images including the first medical image and the second medical image.

15. A scroll cine system as defined in claim 13, wherein the selected indication is the second indication.

16. A scroll cine system as defined in claim 13, further comprising:
a temporary memory to store at least one of the medical image stack, the index, or the indications of medical image locations in the medical image stack; and
a thumbnail generator to generate thumbnail images corresponding to medical images in the medical image stack.

17. A scroll cine system as defined in claim 16, wherein the user interface displays a thumbnail image as the indication of the corresponding medical image location in a medical image stack.

18. A scroll cine system as defined in claim 16, wherein the user interface displays thumbnail images adjacent to a scale of corresponding medical images located at the corresponding location in the medical image stack.

19. A scroll cine system as defined in claim 13, wherein the indications including the first indication and the second indication are at least one of a thumbnail image of the corresponding medical image, text describing the location of the corresponding medical image in the stack, an icon positioned within a scale, or an icon position adjacent to the corresponding medical image.

20. A scroll cine system as defined in claim 18, wherein the scale corresponds to at least one of the index of indications or the medical image stack.

21. A scroll cine system as defined in claim 18, wherein the icon position in the scale corresponds to the location of the associated indication in the medical image stack.

22. A scroll cine system as defined in claim 18, wherein the icon position adjacent to the corresponding medical image corresponds to the location of the associated indication in the medical image stack.

23. A scroll cine system as defined in claim 18, wherein text describing the location of the corresponding medical image in the stack includes at least one of a slice location of the medical image in the medical image stack, an image number of the medical image, a series number of the medical image, or the frame number of the medical image.

24. A scroll cine system as defined in claim 13, wherein the user interface displays an icon indicating that the functionality of scrolling through the index is active.

25. A scroll cine system as defined in claim 13, wherein the medical image stack is a group of medical images generated during one or more medical imaging procedures.

26. A machine accessible medium having instructions stored thereon that, when executed, cause a machine to:
generate an index of indications of medical image locations in a medical image stack including a first indication of a first medical image location in a medical image stack and a second indication of a second medical image location in the medical image stack;
display in a user interface the first medical image and the first indication of the first medial image location within the medical image stack;
receive a scroll input and scroll through the index displaying indications of medical image locations in the image stack until the second indication of the second medical image is displayed;
receive a selection of the second indication of the second medical image; and
display in the user interface the second medical image and the second indication of the second medical image location within the medical image stack.

27. A machine accessible medium as defined in claim 26, wherein the indications including the first indication and the second indication are at least one of a thumbnail image of the corresponding medical image, text describing the location of the corresponding medical image in the stack, an icon positioned within a scale, or an icon position adjacent to the corresponding medical image.

28. A machine accessible medium as defined in claim 27, wherein the scale corresponds to at least one of the index of indications or the medical image stack.

29. A machine accessible medium as defined in claim 27, wherein the icon position in the scale corresponds to the location of the associated indication in the medical image stack.

30. A machine accessible medium as defined in claim 29, wherein the machine readable instructions cause the machine to display thumbnail images adjacent to the scale of corresponding medical images located at the corresponding location in the medical image stack.

31. A machine accessible medium as defined in claim 27, wherein the icon position adjacent to the corresponding medical image corresponds to the location of the associated indication in the medical image stack.

32. A machine accessible medium as defined in claim 27, wherein text describing the location of the corresponding medical image in the stack includes at least one of a slice location of the medical image in the medical image stack, an image number of the medical image, a series number of the medical image, or the frame number of the medical image.

33. A machine accessible medium as defined in claim 26, wherein the machine readable instructions cause the machine to upon generating the index, display in a user interface an icon indicating that the functionality of scrolling through the index is active.

34. A machine accessible medium as defined in claim 26, wherein the medical image stack is a group of medical images generated during one or more medical imaging procedures.

35. A machine accessible medium as defined in claim 26, wherein the indication of the medical image location is displayed at least one of adjacent to a medical image in the user interface or on top of a medical image in the user interface.

36. A method of indicating a medial image location in an image stack, the method comprising:
generating an index of indications of medical image locations in a medical image stack of a primary medical image including a first indication of a first medical image location in a medical image stack and a second indication of a second medical image location in the medical image stack;
receiving a selection of the location on the primary medical image of the first medical image;
displaying in a user interface the first medical image and the first indication of the first medial image location within the primary image;
receiving a scroll input and scrolling through the index displaying indications of medical image locations in the primary image until the second indication of the second medical image is displayed;
receiving a selection of the second indication of the second medical image; and
displaying in the user interface the second medical image and the second indication of the second medical image location within the primary image.
